(19) United States
(12) Patent Application Publication
(10) Pub. No.: US 2011/0077664 A1
(43) Pub. Date: Mar. 31, 2011

(54) DEVICE FOR PROCESSING DERMAL TISSUE

(75) Inventors: Olaf Hyon Schulz, Lakeland, TN (US); Lauren Ann Hellmann, Parsippany, NJ (US); David Bradford Harness, Eads, TN (US); Nicholas John Byrne, Memphis, TN (US)

(73) Assignee: WRIGHT MEDICAL TECHNOLOGY, INC., Arlington, TN (US)

(21) Appl. No.: 12/568,263

(22) Filed: Sep. 28, 2009

(51) Int. Cl. A61B 17/322

(52) U.S. Cl. 606/132

(57) ABSTRACT

A device for processing harvested dermal tissue includes a body having a cutting head portion and a handle portion extending from the cutting head portion. The cutting head portion defines a chamber. A cutting assembly is disposed in the chamber and includes a plurality of blades and a flexible spacer member disposed on the axle such that the blades rotate as the axle rotates. Each of the plurality of blades is separated from an adjacent blade by a spacer disposed on the axle. A grill assembly is coupled to the body and includes a grill and a grill cap. The grill has a plurality of spaced apart slots sized and configured to receive the blades and maintain the blades in a position with respect to the body. The grill cap is sized and configured to couple the grill to the cutting head portion of the body.

### FIELD OF THE DISCLOSURE

The disclosed system relates a device for processing dermal tissue that has been harvested or otherwise removed from a patient. More specifically, the disclosed system relates to a device for cutting dermal tissue into particles that are suitable for transplantation into a wound on the patient.

### BACKGROUND OF THE INVENTION

Skin grafting involves removing of a thin slice of dermal tissue from a donor site on a patient. The harvested tissue slice is then used to cover the site of a wound, which is typically a non-self-healing wound or a burn. The harvested tissue is frequently processed before it is applied to the recipient wound site to expand the area of the harvested tissue in order to minimize the amount of tissue that needs to be harvested from the donor site.

A common process called “meshing” creates a number of small, non-connected cuts in the slice of tissue. The harvested and processed tissue can then be stretched until it has the appearance of a mesh or net. Other processing methods include cutting the tissue into particles with knives, blades, or scissors.

Conventional devices for processing dermal tissue utilize a drum carrying a plurality of parallel blades. The strip of tissue is placed on the cutting surface, and the device is activated thereby rotating the drum and bringing the blades into contact with the underlying cutting surface. The tissue is manually translated across the cutting surface to enable the blades to slice the tissue into fine strips. The strips of tissue may then be repositioned on the cutting surface to enable the blades to cut the strips into individual particles. Unfortunately, particles may accumulate in the interstices between adjacent blades. This accumulation of tissue necessitates manual removal using a spatula or the like instrument.

Some devices, such as the device disclosed in U.S. patent application Ser. No. 10/789,620 titled “Method and Apparatus for Processing Dermal Tissue”, attempt to solve this problem by including separators having tines that interdigitate with blade tips. However, such devices are expensive to manufacture as they include numerous individual components. Additionally, these devices do not include any features that prevent them from being sterilized and reused by a hospital, which increases the chances of spreading diseases or infections if the device is not thoroughly sterilized.

Accordingly, a device for processing harvested dermal tissue that is inexpensive to manufacture and is easy and effective to use is desirable.

### SUMMARY OF THE INVENTION

In some embodiments, a device for processing harvested dermal tissue is provided that includes a body having a cutting head portion with a handle portion extending from the cutting head portion. The cutting head portion defines a chamber. A cutting assembly is disposed in the chamber and includes a plurality of blades and a flexible spacer member disposed on an axle such that the blades rotate as the axle rotates. Each of the plurality of blades is separated from an adjacent blade by a spacer disposed on the axle. A grill assembly is coupled to the body and includes a grill and a grill cap. The grill has a plurality of spaced apart slots sized and configured to receive and maintain the blades in position with respect to the body. The grill cap is sized and configured to couple the grill to the cutting head portion of the body.

In some embodiments, another device for processing harvested dermal tissue is provided that includes a body having a cutting head portion and a handle portion, a second handle, a cutting assembly, and a grill assembly. The handle portion extends from the cutting head portion, which defines a cavity. The second handle is pivotally coupled to the cutting head portion of the body such that it pivots between a collapsed position and an extended position. The cutting assembly is rotatably disposed within the chamber of the body. The cutting assembly includes a plurality of blades disposed on an axle such that as the axle rotates about its axis each of the plurality of blades rotates about the same axis. Adjacent blades of the plurality of blades are spaced apart by a spacer member disposed on the axle. At least one flexible spacer member is disposed on the axle adjacent to one of a pair of retaining clamps. A pair of bearings are disposed on a portion of the axle and are disposed within a pair of slots defined in inner walls of the cutting head portion of the body. The grill assembly includes a grill and a grill cap. The grill has a plurality of spaced apart slots. Each of the spaced apart slots is sized and configured to receive a respective one of the plurality of blades. The grill cap is sized and configured to couple the grill to the cutting head portion of the body and deform during an autoclaving process.

The foregoing and other aspects will be apparent from the following description of the preferred embodiments. In the description, reference is made to the accompanying drawings which form a part hereof, and in which there is shown by way of illustration, and not limitation, a preferred embodiment. Such embodiment does not necessarily represent the full scope of the invention, and reference must therefore be made to the claims herein for interpreting the scope of the invention and its equivalents.

### BRIEF DESCRIPTION OF THE SEVERAL VIEWS OF THE DRAWINGS

These and other features and advantages of the present invention will be more fully disclosed in, or rendered obvious by the following detailed description of the preferred embodiments of the invention, which are to be considered together with the accompanying drawings wherein like numbers refer to like parts and further wherein:

FIG. 1 is an isometric view of an improved device for processing harvested dermal tissue;

FIG. 2 is an elevated side view of the device for processing harvested dermal tissue illustrated in FIG. 1;

FIG. 3 is an elevated top side view of the device for processing harvested dermal tissue illustrated in FIG. 1.

FIG. 4 is an elevated side view of a body of the device for processing harvested dermal tissue illustrated in FIG. 1;

FIG. 5 is an elevated bottom-side view of the body of the device for processing harvested dermal tissue illustrated in FIG. 4;

FIG. 6 is an elevated top-side view of the body of the device for processing harvested dermal tissue illustrated in FIG. 4;

FIG. 7 is a elevated view of the cutting head of the body of the device for processing harvested dermal tissue illustrated in FIG. 4;

FIG. 8 is a sectional view taken along the longitudinal axis of the body of the device for processing harvested dermal tissue illustrated in FIG. 4;

FIG. 9 is a detail view of the device for processing harvested dermal tissue illustrated in FIG. 8;

FIG. 10 is an elevated side view of a handle of the device for processing harvested dermal tissue illustrated in FIG. 1;

FIG. 11 is an elevated bottom-side view of the handle of the device for processing harvested dermal tissue illustrated in FIG. 10;

FIG. 12 is an elevated top-side view of the handle of the device for processing harvested dermal tissue illustrated in FIG. 10;

FIG. 13 is an elevated front-end view of a blade sub-assembly of the device for processing harvested dermal tissue illustrated in FIG. 1;

FIG. 14 is an elevated view of an axle of the blade sub-assembly illustrated in FIG. 13;

FIG. 15 is an elevated side view of the axle illustrated in FIG. 14;

FIG. 16 is an elevated side view of a blade of the blade sub-assembly illustrated in FIG. 13;

FIG. 17 is an elevated view of the blade illustrated in FIG. 165;

FIG. 18 is a detail view of the blade tip of the blade illustrated in FIG. 17;

FIG. 19 is an elevated side view of a spacer member of the blade sub-assembly illustrated in FIG. 13;

FIG. 20 is an elevated view of the spacer member illustrated in FIG. 19;

FIG. 21 is an elevated side view of a flexible spacer member of the blade sub-assembly illustrated in FIG. 13;

FIG. 22 is an elevated view of the flexible spacer member illustrated in FIG. 21;

FIG. 23 is an elevated side view of a clamp of the blade sub-assembly illustrated in FIG. 13;

FIG. 24 is an elevated view of the clamp illustrated in FIG. 23;

FIG. 25 is an elevated side view of a bearing of the blade sub-assembly illustrated in FIG. 13;

FIG. 26 is an elevated view of the bearing illustrated in FIG. 25;

FIG. 27 is an elevated view of a grill sub-assembly of the device for processing harvested dermal tissue illustrated in FIG. 1;

FIG. 28 is an elevated view of a grill of the grill sub-assembly illustrated in FIG. 27;

FIG. 29 is an elevated side view of the grill illustrated in FIG. 28;

FIG. 30 is a detail view of the grill illustrated in FIG. 28;

FIG. 31 is an elevated view of a grill cap of the grill sub-assembly illustrated in FIG. 27;

FIG. 32 is a sectional view of the grill cap taken along line 32-32 in FIG. 31;

FIG. 33 is a sectional view of the grill cap taken along line 33-33 in FIG. 31;

FIG. 34 is a detail view of the grill cap illustrated in FIG. 33;

FIG. 35 is an exploded view of the cutting assembly of the device for processing harvested dermal tissue illustrated in FIG. 1;

FIG. 36 is a detail view of the cutting assembly installed in the cutting head portion of the device for processing harvested dermal tissue illustrated in FIG. 1;

FIGS. 37A-37B illustrate the device for processing harvested dermal tissue illustrated in FIG. 1 in use;

FIG. 38A illustrates the device for processing harvested dermal tissue illustrated in FIG. 1 disposed in an autoclave;

FIG. 38B illustrates the device for processing harvested dermal tissue illustrated in FIG. 1 after being autoclaved;

FIG. 38C illustrates the grill cap after autoclaving the device for processing harvested dermal tissue illustrated in FIG. 1;

FIG. 39 is a top plan view of one example of a dermatome;

FIG. 40 is a side plan view of the dermatome illustrated in FIG. 39;

FIG. 41 is a top plan view of one example of a cutting surface; and

FIG. 42 is a side plan view of the cutting surface illustrated in FIG. 41.

### DETAILED DESCRIPTION OF THE PREFERRED EMBODIMENTS

This description of preferred embodiments is intended to be read in connection with the accompanying drawings, which are to be considered part of the entire written description. The drawing figures are not necessarily to scale and certain features of the invention may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship.

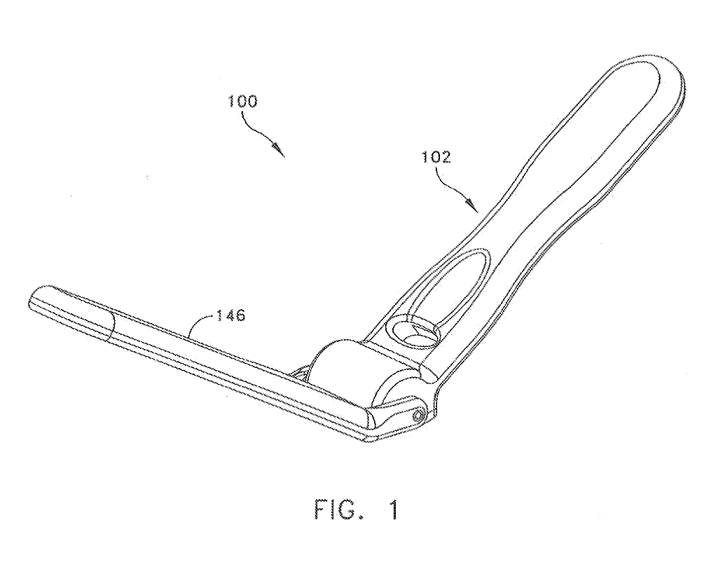

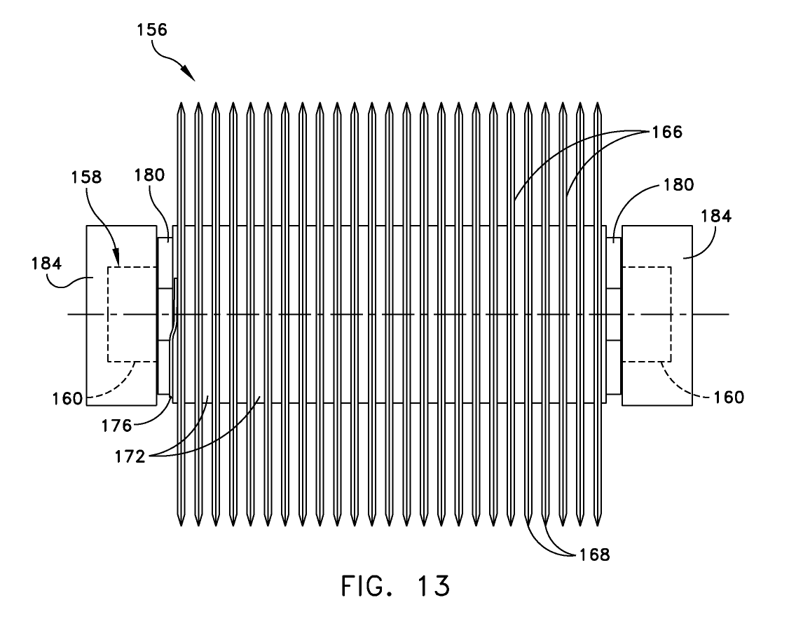

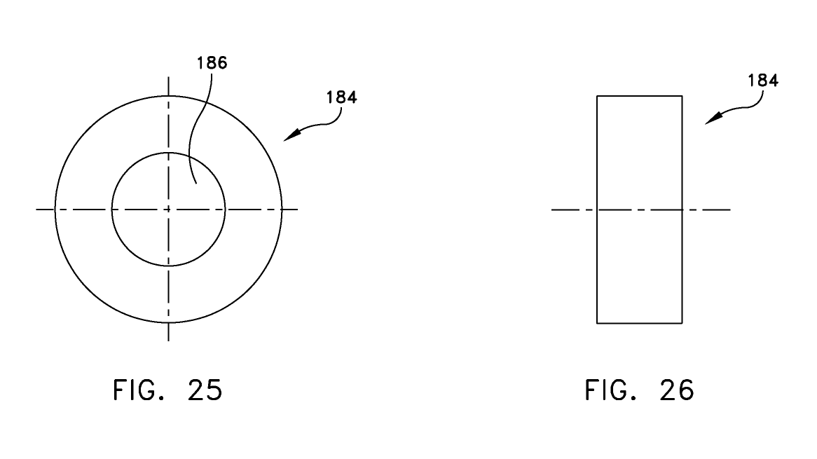

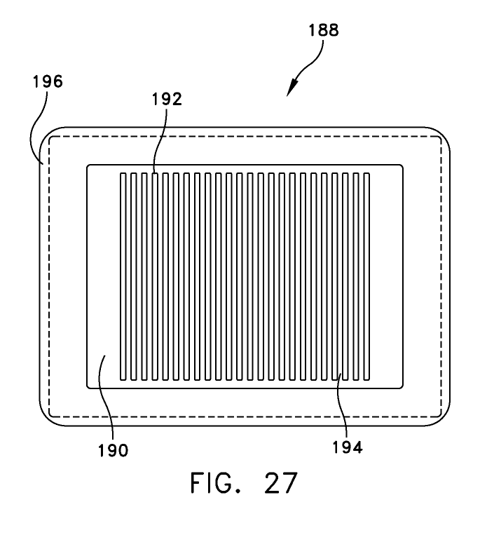

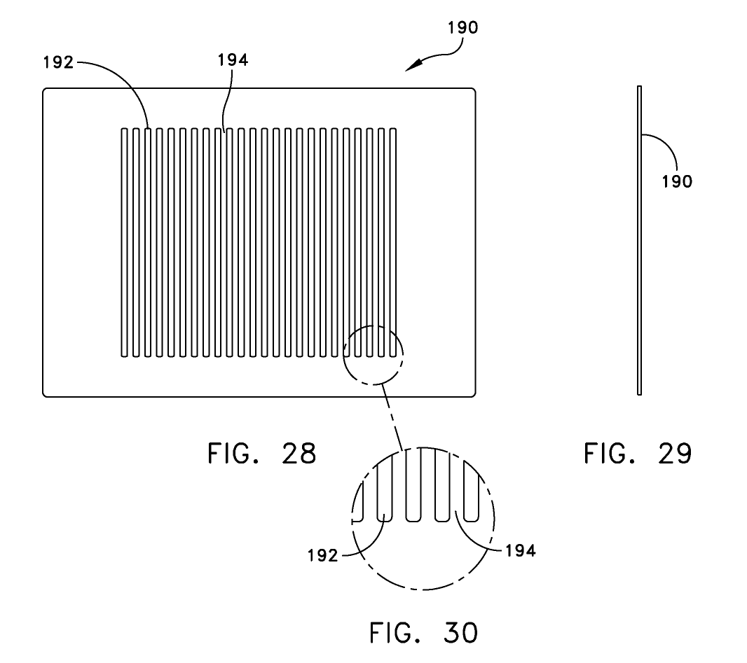

Referring now to FIGS. 1-38, a device for processing harvested dermal tissue is provided that may be manufactured at a lower cost compared to conventional devices, and offers enhanced safety by ensuring the device is used only once. Referring to FIG. 1, a disposable, handheld device 100 for processing harvested dermal tissue includes a device body 102 having a cutting head portion 104 and a handle portion 128 extending from the cutting head portion 104. The cutting head portion 104 defines a chamber 106 sized and configured to receive and house a blade sub-assembly 156 (FIG. 13) including a plurality of spaced apart blades 166 through opening 108. Blade sub-assembly 156 may be secured inside the cutting head portion 104 by a grill sub-assembly 188 (FIG. 27). A second handle 146 (FIG. 1) is pivotally coupled to the cutting head portion 104 of the device body 102.

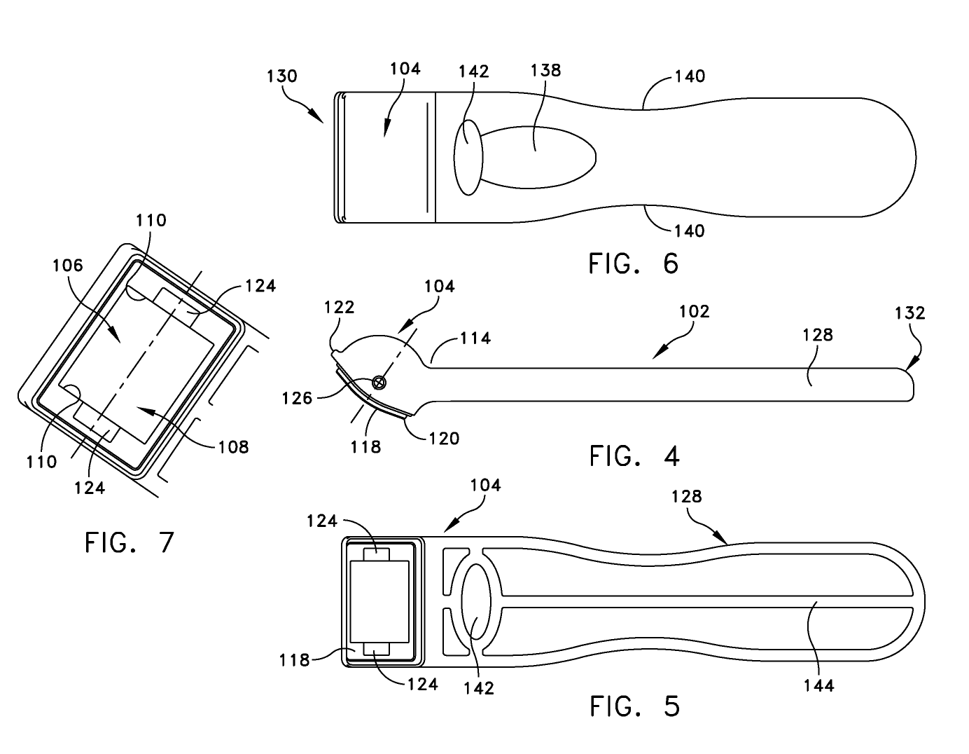

Referring to FIG. 4, cutting head portion 104 of the device body 102 often has convex top and bottom surfaces 114, 118. One skilled in the art will appreciate that cutting head portion 104 may have a variety of other geometric shapes. Referring to FIGS. 4 and 9, bottom surface 118 of device body 102 may include a ridge 120 around the perimeter of opening 108. Additionally, a stop or flange 122 may project outwardly from top surface 114 of device body 102 adjacent to opening 108.

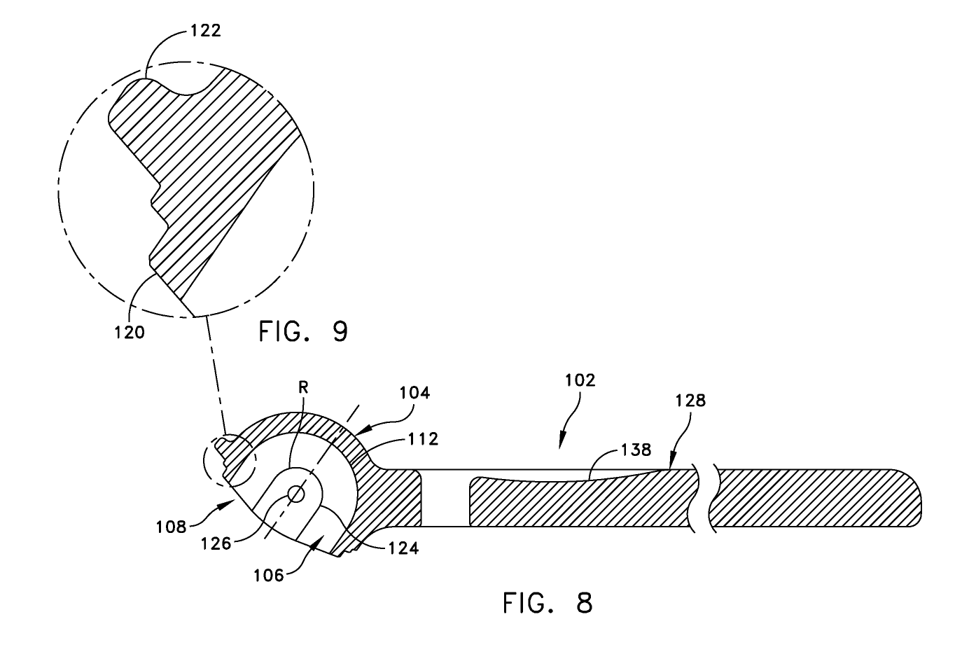

Referring to FIGS. 7 and 8, chamber 106 of cutting head portion 104 may have a concave top wall 112 and straight side walls 110. Each of side walls 110 of chamber 106 may include a slot 124 that projects inwardly from bottom surface 118 of device body 102, terminating at radius R (FIG. 8). In some embodiments, slots 124 have a width of approximately 8.2 mm (approximately 0.32 inch) and a depth of approximately 10.4 mm (approximately 0.41 inch) although one skilled in the art would understand that slots 124 may have other dimensions that enable receipt of bearings 184 of cutting assembly 154 as will be further described in relation to embodiments of the invention. Each of slots 124 may define a blind or through hole 126 that extends partially or through side walls 110 of cutting head portion 104. Holes 126 may be sized and configured to receive a dowel pin 206 or other coupling device.

Handle portion 128 of device body 102 extends from cutting head portion 104. Preferably, handle portion 128 extends from the device body at an angle with respect to an axis defined by slots 124 (FIG. 8) of from between 0 and 90 degrees, and preferably between 20 and 70 degrees, and more preferably between 50 and 60 degrees. Referring to FIGS. 5 and 6, handle portion 128 may include a slight taper near its proximal end 130 adjacent to cutting head portion 104 and terminate in a curved distal end 132. A shallow depression 138 may be formed on top surface 134 of handle portion 128 adjacent to a view port 142 (FIGS. 6 and 8) so as to provide an ergonomic shape. A stiffener rib 144 may extend longitudinally along the length of handle portion 128. Referring to FIG. 5, stiffener rib 144 may be disposed on the underside of handle portion 128 approximately along its center to provide additional structural integrity to handle portion 128 while maintaining light weight and keeping to a minimum the amount of raw materials necessary to fabricate device body 102. Device body 102 may be injection molded, machined, or otherwise fabricated from polyethylene, polypropylene, or any other polymeric, plastic, or metallic material suitable for injection molding, machining, and medical device applications.

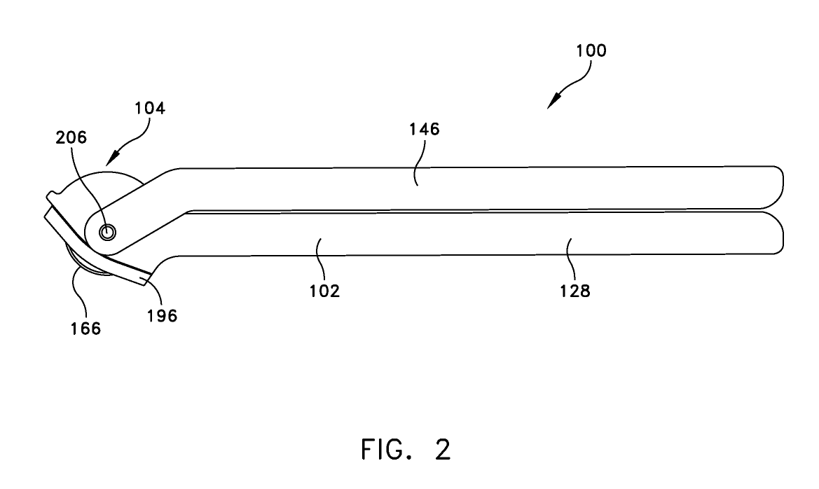

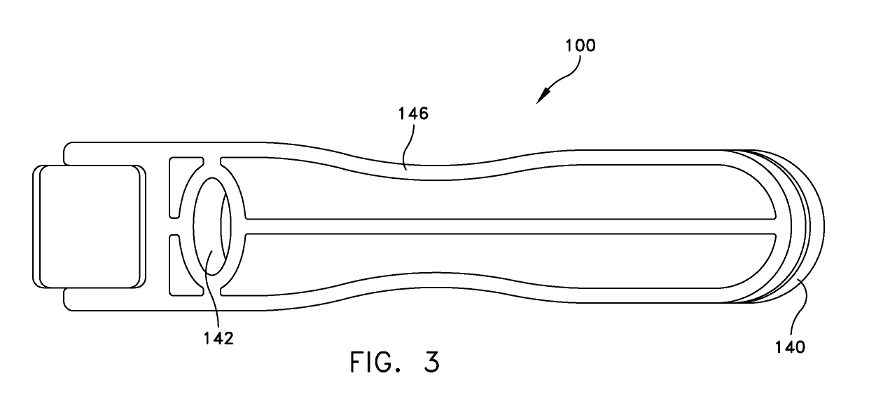

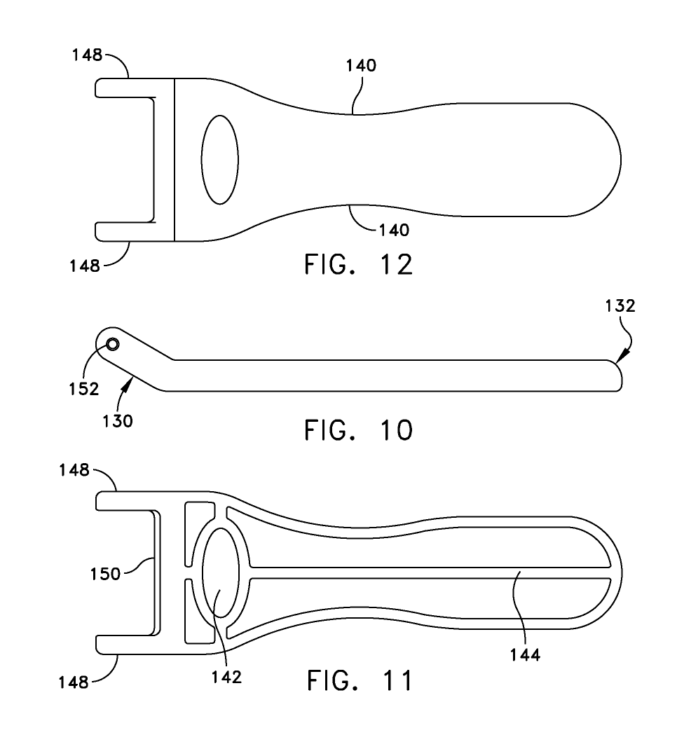

In some embodiments, the device 100 may include a second handle 146, which may also be injection molded, machined, or otherwise fabricated from polyethylene, polypropylene, polymeric, plastic, or metallic material such that it has a similar shape to handle portion 128 of device body 102. However, one skilled in the art will appreciate that handle 146 may also have a shape that substantially differs from handle portion 128. Descriptions of the features of second handle 128 that are similar or identical to the features of the handle portion 128 are not repeated here. Referring to FIGS. 10-12, handle 146 includes a pair of arms 148 that extend from proximal end 130 of handle 146 at an angle with respect to an axis defined by the longitudinal portion of handle 146. Arms 148 are sufficiently spaced apart to receive cutting head portion 104 of device body 102 therebetween. A flange or stop 150 may extend from proximal end 130 of handle 146 between arms 148. A blind or through hole 152 may be formed near the end of each of arms 148 for pivotally coupling handle 146 to cutting head portion 104 of device 100 (FIG. 10). As best seen in FIG. 3, viewing ports 142 defined by each of handles 128, 146 are located on each of handles 128, 146, such that they do not normally align with one another when device 100 is in the folded position, e.g., when handles 128, 146 are disposed in abutting relationship (FIGS. 2 and 3). Locating viewing ports 142 such that they do not align with one another when in a folded position encourages a physician or user of device 100 to employ the device in an open or extended position (FIG. 1).

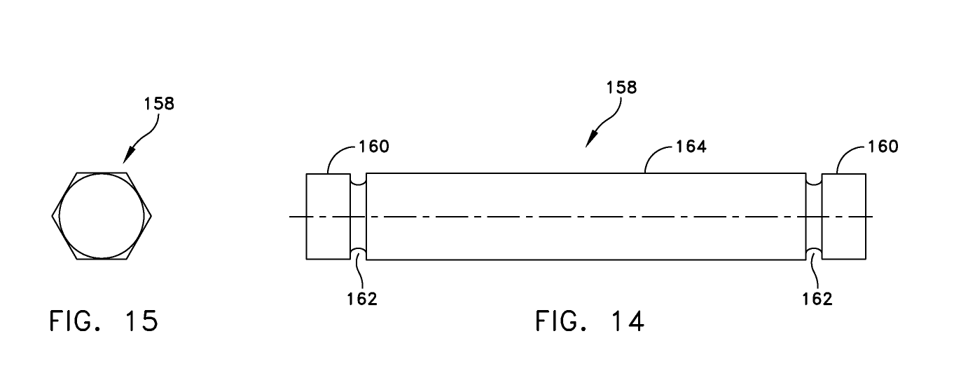

The cutting assembly 154 includes a blade sub-assembly 156 and a grill sub-assembly 188. Turning now to FIGS. 13 and 14, blade sub-assembly 156 comprises an arbor or axle 158, a plurality of rotary blades 166 and spacers 172 alternately disposed on axle 158, one or more flexible spacer members 176, a pair of clamps 180, and a pair of bearings 184. Axle 158 may include a first region 160 having a substantially circular cross-sectional geometry and an undercut region 162 also having a substantially circular cross-sectional geometry, but having a smaller diameter than the diameter of first region 160. A central region 164 extending between regions 160 and 162 may have a polygonal cross-sectional geometry. In some embodiments, axle 158 has an overall length of approximately 1.012 inches with a diameter of first region 160 being approximately 4 mm (approximately 0.16 inch), which may be the same as the width of polygonal region 164. In some embodiments, the first cylindrical region 160 may have a diameter that is less than the thickness of the polygonal region 164 to facilitate the loading of the blades 166 onto the axle 158. Under-cut region 162 may have a diameter of approximately 2.95 mm (approximately 0.12 inch), although one skilled in the art will understand that the three regions of axle 158 may have different dimensions. Polygonal region 164 may be triangular, rectangular, pentagonal, hexagonal, or any other polygon. In some embodiments, polygonal region 164 may have a substantially circular cross-sectional area having a flat, a key, or other like feature for engaging rotary blades 166. Axle 158 may be formed from stainless steel or any other material suitable for use in medical device applications including, but not limited to, titanium, aluminum, and brass. Axle 158 may be injection molded, extruded, machined, or otherwise fabricated as will be understood by one skilled in the art. In alternative embodiments, the axle, 158, blades 166, and spacers 172 monolithically formed by turning down a single piece of material.

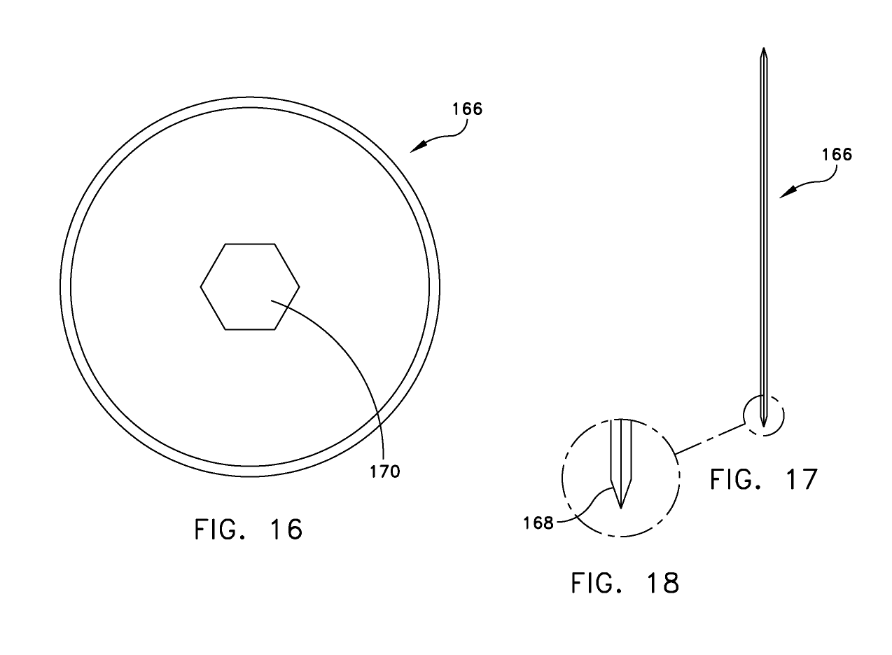

FIGS. 16-18 illustrate one example of a rotary blade 166 where each rotary blade 166 may have a substantially circular outer shape and define a central aperture 170 having a complementary shape with the shape of polygonal region 164 of axle 158. In some embodiments, each rotary blade is formed from stainless steel and has an outer diameter of approximately 18 mm (approximately 0.71 inch) and a thickness of approximately 3 mm (approximately 0.12 inches). Referring to FIG. 18, each blade 166 may have a blade tip 168 having a length of approximately 4.8 mm (approximately 0.19 inches), although the blade tips may have other lengths that are sufficiently long to cut through harvested dermal tissue as will be understood by one skilled in the art. Additionally, the blades 166 may have a variety of configurations including, but not limited to, notches in the blade tips or multiple blade tips per blade that are separated by a channel. Each of blades 166 may define a central key hole or aperture 170 sized and configured to receive polygonal region 164 of axle 158. For example, central aperture 170 may have a hexagonal cross-sectional geometry with a width of approximately 0.160 to slidingly receive polygonal region 164 of axle 158 therein.

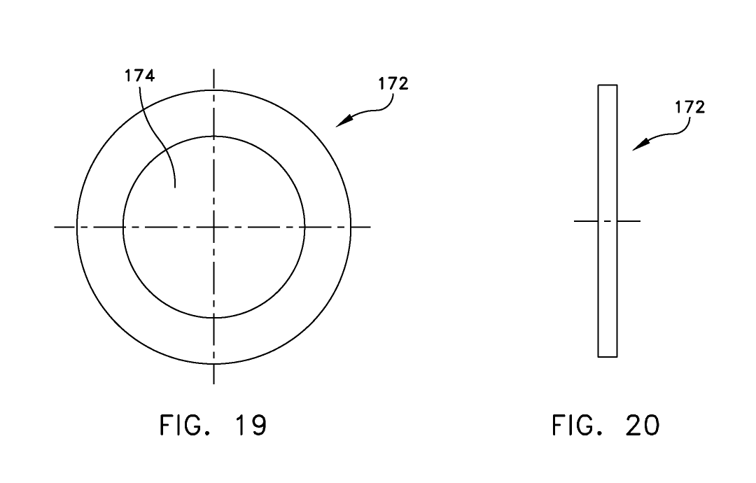

Adjacent rotary blades 166 of blade sub-assembly 156 are separated by spacers or washers 172 (FIGS. 19 and 20). Spacers 172 often have an outer diameter that is less than the outer diameter of rotary blades 166. In some embodiments, spacers 172 may have a diameter of approximately 7.87 mm (approximately 0.31 inch) and a width of approximately 5 mm (approximately 0.2 inch). One skilled in the art would understand that the width of spacers 172 may be adjusted to cut the harvested dermal tissue to the desired width. Additionally, the outer diameter of spacers 172 may also be adjusted depending upon the depth of slots 124 formed in side walls 110 of device body 102 such that spacers 172 do not interfere with the attachment of grill sub-assembly 188, but allow blade sub-assembly 156 to float, e.g., vertically move along slots 124 in cutting head portion 104. Spacers 172 may define an aperture 174 that is often substantially circular (FIG. 19) or may have a shape that is complementary to the cross-sectional shape of polygonal region 164 of axle 158. Spacers 172 may be manufactured from stainless steel, titanium, aluminum, brass, a polymer, plastics, or the like.

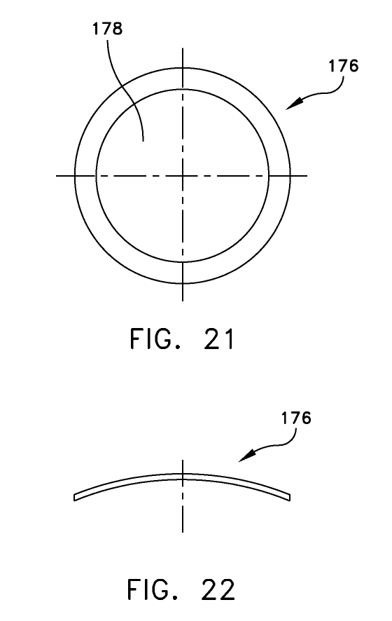

The alternating arrangement of blades 166 and spacers 172 is often secured on axle 158 by a combination of clamps 180. One or more flexible spacer members 176 may be disposed on axle 158 adjacent to one of clamps 180 to compensate for tolerance stack-up of blades 166 and spacers 172 along axle 158. Flexible spacer member 176 may be a wave spring (FIGS. 21 and 22) having a thickness of approximately 0.15 mm (approximately 0.0059 inch), an outer diameter of approximately 6.2 mm (approximately 0.24 inch), and an inner diameter of approximately 5 mm (approximately 0.2 inch). One skilled in the art will understand that the size and shape of the spacer may be adjusted. The inclusion of one or more flexible spacer members 176 between clamps 180 and the arrangement of alternating blades 166 and spacers 172 enables blades 166 and spacers 172 to shift along the axis defined by axle 158. Once device 100 is assembled, blades 166 are held in place by slots 192 in grill 190.

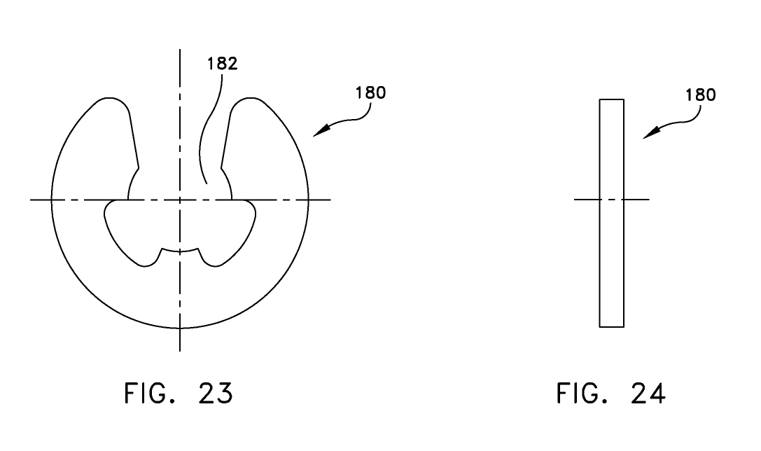

Referring to FIGS. 23 and 24, clamp 180 is sized and configured to engage undercut region 162 of axle 158. In some embodiments, clamp 180 may have an outer diameter of approximately 7.2 mm (approximately 0.28 inch), an inner diameter of approximately 3 mm (approximately 0.12 inch), and a thickness of approximately 0.6 mm (approximately 0.024 inch), although clamp 180 may have other dimensions and shapes. Flexible spacer members 176 and clamp 180 may each be fabricated from stainless steel or any other sufficiently resilient medical-grade materials.

Bearings 184 are sized and configured to be disposed on first region 160 of axle 158 and enable axle 158 to rotate about an axis when blade sub-assembly 156 is disposed in cutting head portion 104 of device body 102. In some embodiments, bearings 184 may be polyethylene bearings having an inner diameter of approximately 4 mm (approximately 0.16 inch), an outer diameter of approximately 8 mm (approximately 0.31 inch), and a thickness of approximately 3 mm (approximately 0.12 inch). One skilled in the art would understand that other polymers may be used to form these parts. Polyethylene bearings 184 are less expensive and have a longer shelf-life compared to ball bearings that are implemented in conventional devices for processing harvested dermal tissue. Additionally, polyethylene or soft polymer bearings 184 are more susceptible to sterilization compared to conventional ball bearings implemented in dermal processing devices.

FIGS. 27-30 illustrate one example of a grill sub-assembly 188 including a grill 190 and a frame or cap 196. Grill 190 often includes a plurality of parallel, spaced apart slots 192 separated by parallel, spaced apart bars 194. Each of slots 192 is sized and configured to receive a portion of a blade 166 yet provide sufficient clearance such that the blade may rotate about an axis defined by the axle 158 as the device 100 is translated across a cutting surface 216. In one embodiment, slots 192 have a length of approximately 16.5 mm (approximately 0.65 inch), a width of approximately 0.4 mm (approximately 0.016 inches), and be spaced apart from adjacent slots 192 by approximately 8.5 mm (approximately 0.33 inch) measured from center-to-center. Each end of slots 192 may include a radius of approximately 0.135 mm (approximately 0.0053 inch). Grill 190 may be formed from a hardened stainless steel, e.g., ASTM A276 type 302 stainless steel or ASTM A167 type 304 stainless steel. Hardening the stainless steel provides additional rigidity and resilience that reduces the likelihood of bars 194 deforming and causing blades 166 to lock up as blades 166 rotate about axle 158.

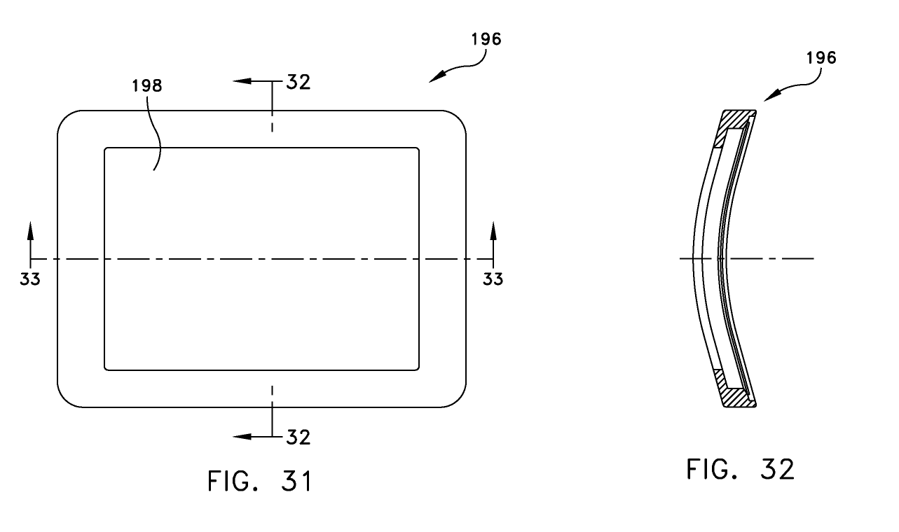

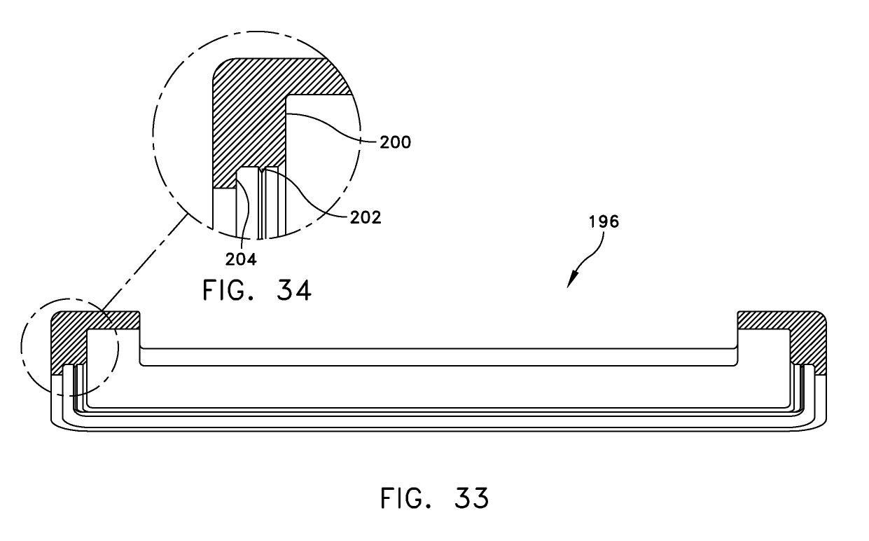

Referring to FIGS. 31-34, a grill frame or cap 196 has a substantially rectangular shape defining a rectangular window 198. Grill frame 196 curves along its width (FIGS. 31-32). The curvature of grill frame 196 complements the curvature of bottom surface 118 of cutting head portion 104 of device body 102. Grill frame 196 includes a recessed shelf 200 sized and configured to support grill 190 and detent or energy director 202 spaced apart from first shelf surface 200. The grill 190 and grill frame 196 are dimensioned such that the grill 190 may be press fit against the recessed shelf 200. The energy director 202 (FIGS. 33 and 34) is sized and dimensioned such that it melts during an ultrasonic welding process to secure the grill sub-assembly 188 to the cutting head portion 104 of the device body 102. In some embodiments, the energy director 202 inwardly extends from the interior portion of the grill frame 196 approximately 0.13 mm (approximately 0.005 inches) in the form of an equilateral triangle, although one skilled in the art will understand that the energy director may have various sizes and geometries. Grill frame 196 includes a second shelf surface 204 that is sized and configured to complement ridge 120 disposed around opening 108 located in bottom surface 118 of device body 102. Grill cap 196 may be injection molded from polyethylene, polypropylene, or any other material suitable for injection molding and that may be ultrasonically welded to device body 102.

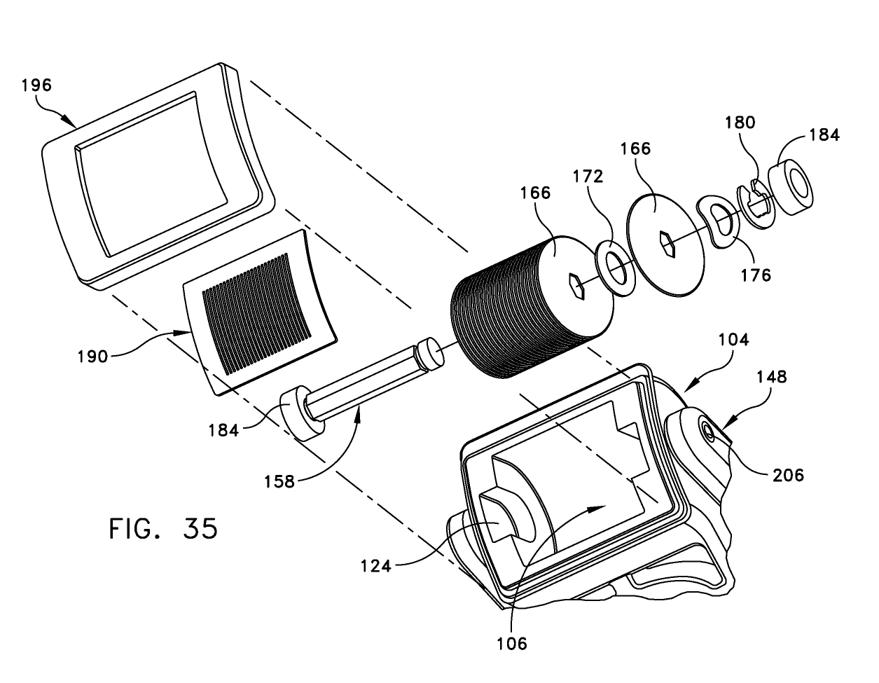

To assemble device 100, second handle 146 is coupled to cutting head portion 104 of device body 102 by inserting a pin 206 into holes 152 formed in arm 148 of second handle 146 and into holes 126 in cutting head portion 104. The assembly of blade sub-assembly 156 and grill sub-assembly 188 is illustrated in the exploded assembly view in FIG. 35. The blade sub-assembly 156 may be formed by clamping a clamp 180 onto one of undercut regions 162 of axle 158. A first flexible spacer member 176 may be slid along axle 158 until it abuts clamp 180. A first blade 166 may be slid onto axle 158 followed by a first spacer 172 or vice versa. A second blade 166 and a second spacer 172 may then be slid onto axle 158. Additional blades 166 and spacers 172 are alternately slid onto axle 158 until a predetermined number of blades 166 have been placed on axle 158. A second flexible spacer member 176 may then be slid onto axle 158 and secured by clamping a second clamp 180 onto undercut region 162 of axle 158.

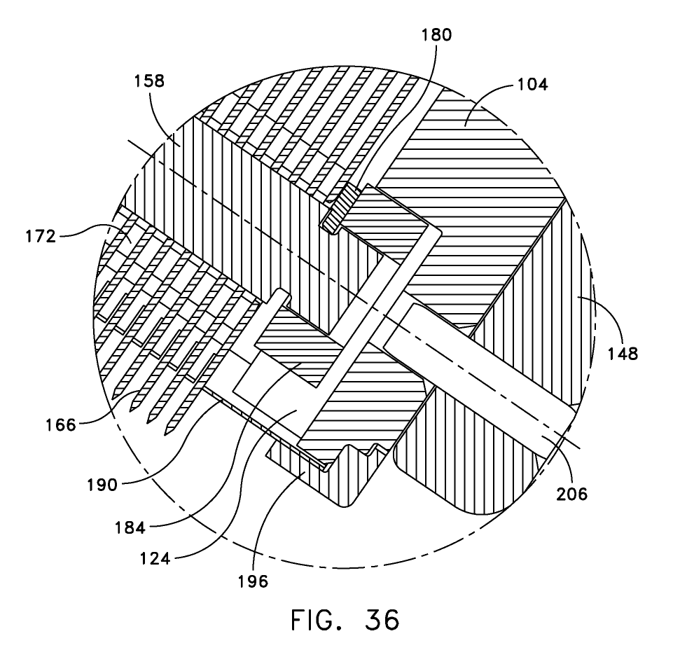

First and second bearings 184 are slid onto first region 160 of axle 158 and blade sub-assembly 156 may then be disposed in cutting head portion 104 of device body 102 by sliding bearings 184 into corresponding slots 124 formed in side walls 110 of cutting head portion 104 of device body 102 (FIG. 36). Grill sub-assembly 188 is assembled by inserting grill 190 into grill frame 196 until the edges of grill 190 are received past detent 202. When installed, grill 190 has a curvature that matches the curvature of grill frame 196 (FIG. 32). Grill sub-assembly 188 is then placed over blade sub-assembly 156 disposed within cutting head portion 104 of device body 102 such that blades 166 are received within slots 192 of grill 190. Second shelf surface 204 is disposed adjacent to ridge 120 around opening 108 located in bottom surface 118 of device body 102. Grill frame 196 may be ultrasonically welded to cutting head portion 104 of device body 102 to secure blade sub-assembly 156 inside chamber 106 of device body 102. In some embodiments, grill frame 196 may be secured to the cutting head portion 104 of device body 102 by using an adhesive or glue or by using set screws or other attachment means as will be understood by one skilled in the art.

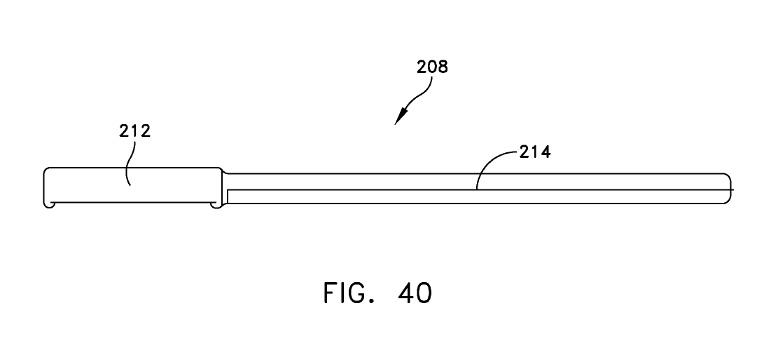

Device 100 for processing harvested dermal tissue may be sold in a kit including a dermatome 208 and a cutting mat 216 (FIGS. 39-42). Dermatome 208 may include a razor blade 210 that is coupled to a housing 212 having a handle 214. Razor blade 210 is secured to housing 212 such that blade 210 extends below a surface of housing 212 by a predetermined distance that corresponds to the desired thickness of dermal tissue to be harvested from a patient.

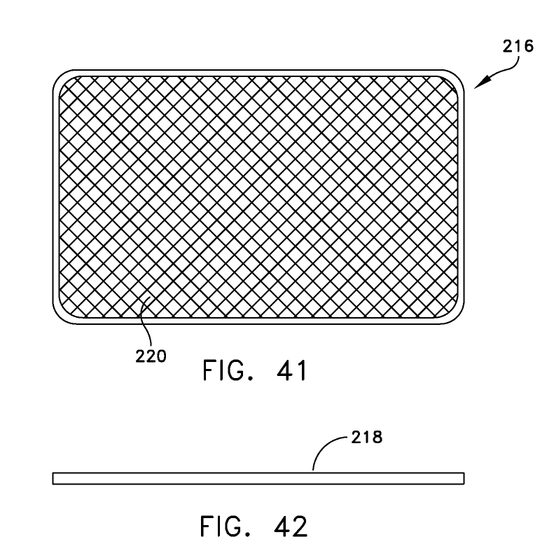

The cutting mat 216 (FIGS. 41 and 42) is preferably fabricated from polypropylene although other materials including, but not limited to, metals and polyethylene may be used. The upper and lower surfaces 218 may include a plurality of convex protrusions 220 or other structures for increasing the friction of top and/or bottom surfaces 218 of cutting mat 216 such as, for example, pyramidal protrusions, concave dimples, cross-hatching, or the like.

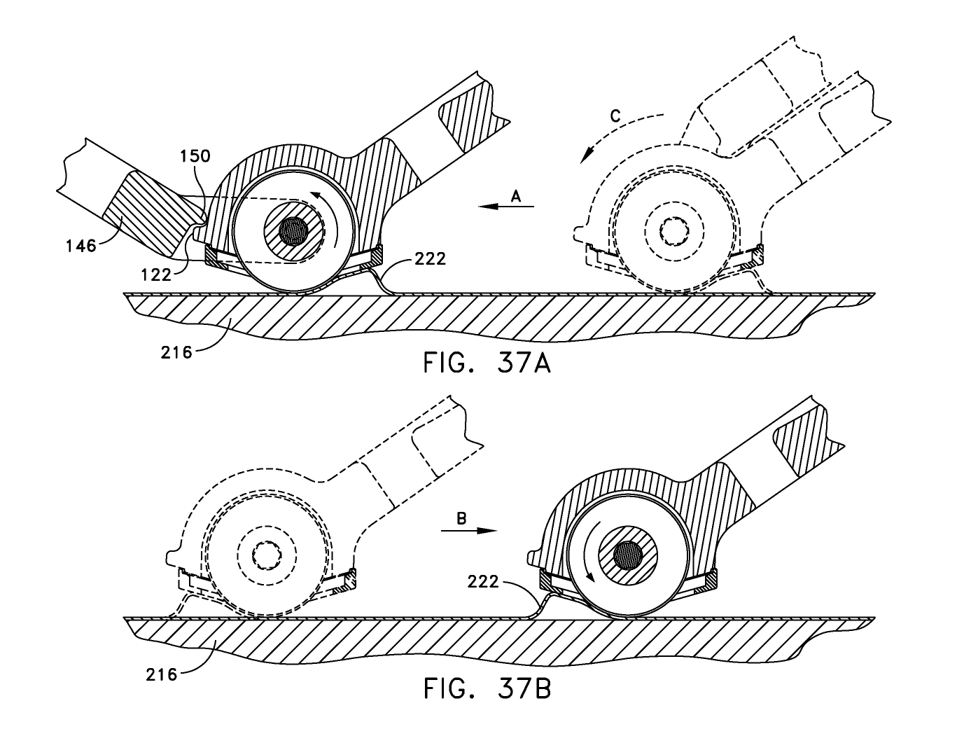

Referring FIGS. 37A-37B, the method of using device 100 for processing harvested dermal tissue includes placing a slice of harvested dermal tissue on a surface 218 of cutting mat 216. The dermal tissue may be harvested using a dermatome 208 (FIG. 39). With the dermal tissue disposed on cutting mat 216, the user may pivot second handle 146, if the device 100 includes a second handle, from the substantially collapsed position in which the second handle is disposed adjacent to handle portion 128 of device body 102 to the extended position as indicated by the arrow C in FIG. 37A. In the extended position, the flange or stop 150 disposed between arms 148 of handle 146 engages flange or stop 122 that extends from top surface 114 of cutting guide portion 104 of device body 102.

The user may then grip device 100 with both hands, e.g., one hand on handle portion 128 and a second hand on handle 146, and arrange device 100 such that blades 166 contact cutting mat 216. The user may then move device 100 in a first direction (arrow A in FIG. 37A or arrow B in FIG. 37B) while maintaining blades 166 engaged with cutting mat 216. Note that the second handle 146 is not shown in FIG. 37B to simplify the figure. As device 100 is moved, the engagement of blades 166 with cutting mat 216 causes blades 166 and axle 158 to rotate. The user may continue to apply a downward force on handles 128, 146 of the device while moving device 100 in the first direction until blades 166 engage harvested dermal tissue 222. Blade tips 168 slice harvest dermal tissue 222 as the user continues to move device 100 in the first direction. Bars 194 of grill 190 disposed between adjacent blades 166 prevent sliced dermal tissue from wrapping around spacers 172 between blades 166.

After passing once through the harvested dermal tissue and creating sliced dermal tissue, the user may repeat the steps described above to further slice the tissue at an angle with respect to the initial slice. For example, the user may pass device 100 through the dermal tissue orthogonally with respect to the direction of the initial slice to create squares of dermal tissue, at a 45 degree angle to create diamond-shaped pieces of dermal tissue, or at any other angle to create further sliced tissue. Once the harvested dermal tissue has been processed, device 100 may be thrown away. To prevent device 100 from being sterilized and reused, grill fame 196 is often ultrasonically welded to cutting head portion 104 of device body 102 and will deform if the device is autoclaved.

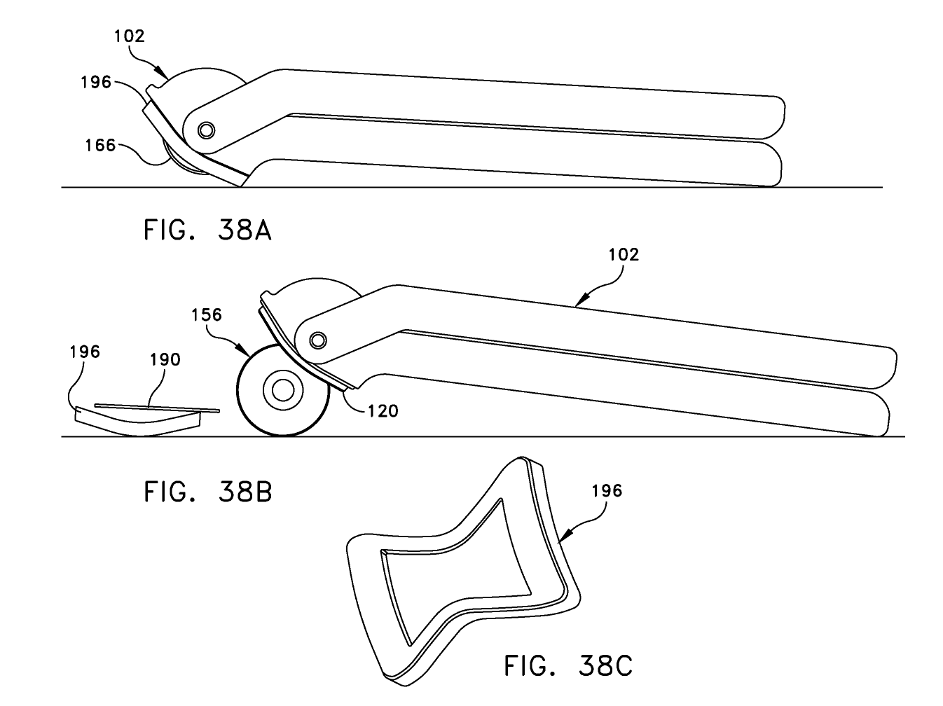

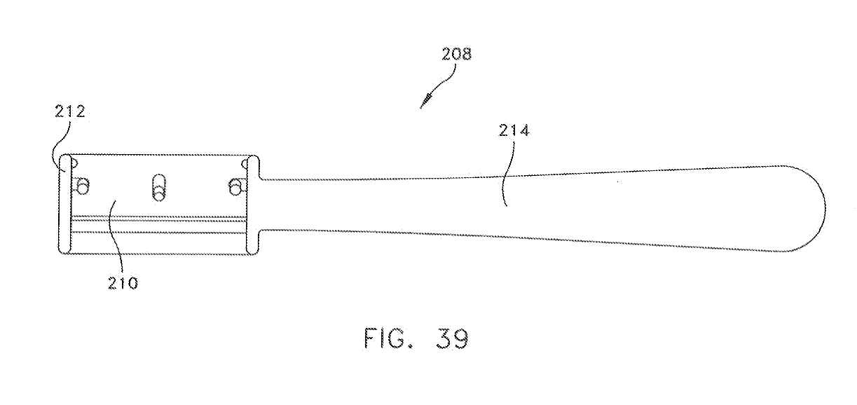

FIGS. 38A-38C illustrate a device 100 before and after it has been autoclaved. As shown in FIG. 38B, cap 196 is severed from the device body 102 after having been autoclaved due to cap 196 being deformed during autoclaving. In this manner, improved device 100 for processing harvested dermal tissue increases the safety by ensuring a single use of the device and preventing hospitals from attempting to sterilize and reuse the device which could lead to cross-contamination as sliced dermal tissue may get stuck between adjacent blades.

The invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass all modifications and alternative arrangements included within the spirit and scope of the invention, as set forth by the appended claims.

What is claimed is:

1. A device for processing harvested dermal tissue, comprising:
a body having a cutting head portion and a handle portion extending from the cutting head portion, the cutting head portion defining a chamber;
a cutting assembly disposed in the chamber, the cutting assembly including a plurality of blades and a flexible spacer member disposed on an axle such that the blades rotate as the axle rotates, each of the plurality of blades separated from an adjacent blade by a spacer disposed on the axle; and
a grill assembly coupled to the body, the grill assembly including:
a grill having a plurality of spaced apart slots sized and configured to receive the blades therein and maintain the blades in a position with respect to the axle, and
a grill cap sized and configured to couple the grill to the cutting head portion of the body.

2. The device of claim 1, wherein the grill cap is sized and configured to deform during an autoclaving process.

3. The device of claim 1, further comprising a pair of bearings disposed on the axle, each of the bearings are received within a respective slot defined by inner walls of the cutting head portion of the body.

4. The device of claim 1, wherein the grill cap is ultrasonically welded to the cutting head portion of the body.

5. The device of claim 1, further comprising:
a second handle pivotally coupled to the body, the second handle configured to pivot between a first position and a second position.

6. The device of claim 5, wherein the handle portion of the body and the second handle each define view ports along their lengths that do not align with one another when the second handle is in the first position in which the second handle abuts the handle portion of the body.

7. The device of claim 5, wherein the cutting head portion includes a protrusion extending from a top surface, the protrusion sized and configured to engage the second handle when the second handle is in the second position.

8. The device of claim 4, wherein the grill cap includes an energy director sized and configured to deform such that the grill cap is severed from the cutting head portion of the body during an autoclave process.

9. The device of claim 1, wherein the grill includes a plurality of substantially parallel, spaced apart bars disposed between the plurality of spaced apart slots.

10. The device of claim 9, wherein the cutting assembly includes a pair of clamps disposed on the axle, the pair of clamps configured to retain the plurality of blades, spacers, and the flexible spacer member disposed on the axle.

11. The device of claim 10, wherein the blades are substantially retained in position along the axle by their engagement with the substantially parallel, spaced apart bars.

12. A device for processing harvested dermal tissue, comprising:
a body including a cutting head portion and a handle portion, the handle portion extending from the cutting head portion, the cutting head portion defining a cavity;
a second handle pivotally coupled to the cutting head portion of the body, the second handle configured to pivot between a collapsed position and an extended position;
a cutting assembly rotatably disposed within the chamber of the body, the cutting assembly including:
a plurality of blades disposed on a first portion axle such that as the axle rotates about its axis each of the plurality of blades rotates about the same axis, adjacent blades of the plurality of blades spaced apart by a spacer member disposed on the axle,
a pair of retaining clamps disposed on the axle,
one flexible spacer member disposed on the axle adjacent to one of the pair of retaining clamps, and
a pair of bearings disposed on a second portion of the axle, the pair of bearings disposed within a pair of slots defined in inner walls of the cutting head portion of the body; and
a grill assembly coupled to the body, the grill assembly including:
a grill having a plurality of spaced apart slots, each of the spaced apart slots sized and configured to receive a respective one of the plurality of blades, and
a grill cap sized and configured to couple the grill to the cutting head portion of the body and deform during an autoclaving process.

13. The device of claim 12, wherein the grill cap is ultrasonically welded to the cutting head portion of the body.

14. The device of claim 12, wherein the handle portion of the body and the second handle each define view ports along their lengths that do not align with one another when the second handle is in the first position in which the second handle abuts the handle portion of the body.

15. The device of claim 12, wherein the cutting head portion includes a protrusion extending from a top surface, the protrusion sized and configured to engage the second handle when the second handle is in the second position.

16. The device of claim 12, wherein the grill cap is sized and configured to deform such that it is severed from the cutting head portion of the body during the autoclave process.

17. The device of claim 12, wherein the grill includes a plurality of substantially parallel, spaced apart bars disposed between the plurality of spaced apart slots.

18. The device of claim 17, wherein the blades are substantially retained in position along the axle by their engagement with the substantially parallel, spaced apart bars.

19. A device for processing harvested dermal tissue, comprising:
a body including a cutting head portion and a handle portion, the handle portion extending from the cutting head portion, the cutting head portion defining a cavity;
a cutting assembly rotatably disposed within the chamber of the body, the cutting assembly including:
a plurality of spaced apart blades disposed on an axle such that as the axle rotates about its axis each of the plurality of blades rotates about the same axis, and
a pair of bearings disposed on a portion of the axle, the pair of bearings disposed within a pair of slots defined in inner walls of the cutting head portion of the body; and
a grill assembly coupled to the body, the grill assembly including:
a grill having a plurality of spaced apart slots, each of the spaced apart slots sized and configured to receive a respective one of the plurality of blades, and
a grill cap sized and configured to couple the grill to the cutting head portion of the body and deform during an autoclaving process.

20. The device of claim 1, wherein the grill cap includes an energy director disposed at least partially around an interior surface of the grill cap, the energy director size and arrange such that it melts to the cutting head portion of the body during an ultrasonic welding process.
